(19) United States
(12) Patent Application Publication
(10) Pub. No.: US 2020/0061131 A1
(43) Pub. Date: Feb. 27, 2020

(54) METHODS AND COMPOSITIONS FOR DETERMINING BACTERIAL INTEGRITY

(71) Applicant: Vedanta Biosciences, Inc., Cambridge, MA (US)

(72) Inventors: Julie Schwedock, Arlington, MA (US); Daniel E. Couto, Concord, MA (US); Scott Michonski, Belmont, MA (US); Ketaki Nade, Cambridge, MA (US)

(73) Assignee: Vedanta Biosciences, Inc., Cambridge, MA (US)

(21) Appl. No.: 16/469,742

(22) PCT Filed: Dec. 15, 2017
(86) PCT No.: PCT/US17/66726

§ 371 Date: Jun. 14, 2019

Related U.S. Application Data
(60) Provisional application No. 62/435,244, filed on Dec. 16, 2016.

(51) Int. Cl. A61K 35/742; C12Q 1/04; C12Q 1/689

(52) U.S. Cl. CPC A61K 35/742; C12Q 1/689; C12Q 1/04

(57) ABSTRACT

The disclosure provides methods and compositions for determining bacterial integrity.

### RELATED APPLICATION

This application claims the benefit under 35 U.S.C. § 119(e) of U.S. provisional application No. 62/435,244, filed Dec. 16, 2016, which is incorporated by reference herein in its entirety.

### FIELD OF THE INVENTION

The disclosure provides methods and compositions for determining bacterial integrity.

### BACKGROUND

The human intestinal microbiome includes a large number of microorganisms. A significant number of these microorganisms are anaerobic bacteria. Compositions that include anaerobic bacteria that originated from the human intestinal microbiome have shown potential in the treatment of human disease (See e.g., Atarashi et al., Nature 500, 232, 2013; Atarashi et al., Cell 163, 1, 2015; Mathewson et al., Nature Immunology 17, 505, 2016). However, anaerobic bacteria are challenging to manufacture because of their sensitivity to oxygen. In addition, any anaerobic bacteria that are to be used for therapeutic applications need to be evaluated for their integrity and readiness for administration. New and improved compositions and methods for determining if anaerobic bacteria have sufficient integrity to be administered are needed therefore.

### SUMMARY

In one aspect, the disclosure provides compositions and methods for determining bacterial integrity.

In some embodiments, the disclosure provides a method for determining the integrity of a bacterial composition, the method comprising: growing the bacterial composition on a selective medium, wherein the bacterial composition comprises a bacterial strain, and wherein if the bacterial composition grows slower on the selective medium than on a non-selective medium, the integrity of the bacterial composition is compromised. In some embodiments of the methods provided herein, the method further comprises growing the bacterial composition on the non-selective medium. In some embodiments, the bacterial composition was lyophilized prior to growing the bacterial composition. In some embodiments, if the integrity of the bacterial composition is compromised, the bacterial composition is not used to prepare a live bacterial product.

In some embodiments, the disclosure provides a method for determining the integrity of a first bacterial composition, the method comprising: growing the first bacterial composition on a selective medium, wherein the first bacterial composition comprises a bacterial strain; growing a second bacterial composition on the selective medium, wherein the second bacterial composition comprises a bacterial strain, and wherein if the first bacterial composition grows slower than the second bacterial composition, the integrity of the first bacterial composition is compromised.

In some embodiments, the disclosure provides a method for determining the integrity of a first bacterial composition, the method comprising: growing the first bacterial composition on a selective medium, growing the first bacterial composition on a non-selective medium, wherein the first bacterial composition comprises a bacterial strain; growing a second bacterial composition on the selective medium, growing the second bacterial composition on the non-selective medium, wherein the second bacterial composition comprises a bacterial strain; wherein if the difference in growth between the selective medium and the non-selective medium for the first bacterial composition is greater than the difference in growth between the selective medium and the non-selective medium for the second bacterial composition, and both the first bacterial composition and the second bacterial composition grow slower on the selective medium than the non-selective medium, the integrity of the first bacterial composition is compromised.

In some embodiments of any of the methods provided herein, the bacterial composition was lyophilized prior to growing the bacterial composition. In some embodiments of any of the methods provided herein, the first bacterial composition was lyophilized prior to growing the first bacterial composition. In some embodiments of any of the methods provided herein, the second bacterial composition was lyophilized prior to growing the second bacterial composition.

In some embodiments of any of the methods provided herein, the selective medium is bile acid media.

In some embodiments, if the integrity of the first bacterial composition is compromised, the first bacterial composition Is not used to prepare a live bacterial product.

In some embodiments of any of the methods provided herein, the first bacterial composition and the second bacterial composition comprise the same bacterial strain. In some embodiments of any of the methods provided herein, the bacterial strain is an anaerobic bacterial strain. In some embodiments, the bacterial strain belongs to the class Clostridia. In some embodiments, the bacterial strain belongs to the family Clostridiaceae. In some embodiments, the bacterial strain belongs to the genus Clostridium. In some embodiments, the bacterial strain is selected from the group consisting of Clostridium bolteae, Anaerotruncus colihominis, Ruminococcus torques, Clostridium symbiosum, Blautia producta, Dorea longicatena, Erysipelotrichaceae bacterium, and Subdolinogranulum spp. In some embodiments of any of the methods provided herein, the bacterial strain is Clostridium bolteae. In some embodiments of any of the methods provided herein, the bacterial strain is Dorea longicatena.

In some embodiments, the bacterial strain comprises a 16S rDNA sequence having at least 97% sequence identity with a nucleic acid sequence selected from the group consisting of SEQ ID NOs: 1-8.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIGS. 1A and 1B show the results of experiments with Dorea longicatena (Strain 6). In FIG. 1A, the bars from left to right are: TSA blood (Fresh), TSA blood+bile (Fresh), TSA blood (Frozen), TSA blood+bile (Frozen), TSA blood (Lyo), TSA blood+bile (Lyo). In FIG. 1B, the bars from left to right are: TSA blood (Fresh), TSA blood+bile (Fresh), TSA blood (Frozen), TSA blood+bile (Frozen), TSA blood (Lyo), TSA blood+bile (Lyo). TSA: Tryptic Soy Agar. Lyo: lyophilized.

FIGS. 2A and 2B show the results of experiments with Clostridium bolteae (Strain 1). In FIG. 2A, the bars from left to right are: PYG (Fresh), TSA blood (Fresh), TSA blood and ox bile (Fresh), PYG (Lyo), TSA blood (Lyo), TSA blood and ox bile (Lyo). In FIG. 2B, the bars from left to right are: TSA blood (Fresh), TSA blood and ox bile (Fresh), TSA blood (Lyo), TSA blood and ox bile (Lyo). PYG: Peptone Yeast Glucose media. TSA: Tryptic Soy Agar. Lyo: lyophilized.

FIGS. 3A and 3B show the results of experiments assessing the viability of the indicated bacterial strains. FIG. 3A shows bacterial viability prior to lyophilization and storage at −80° C. FIG. 3B shows bacterial viability after lyophilization and storage at −80° C. In FIG. 3A, the bars for each of the strains are left: viability on chocolate agar, and right: viability on chocolate agar+ox bile. In FIG. 3B, the bars for each of the strains are left: viability on chocolate agar, and right: viability on chocolate agar+ox bile.

FIGS. 4A and 4B show the results of experiments assessing the viability of bacterial strains following storage at 25° C. FIG. 4A shows bacterial viability following short term storage at 25° C. FIG. 4B shows bacterial viability after one month of storage at 25° C. In FIG. 4A, the bars for each of the strains are, from left to right, initial viability (T0 Viable Titer cfu/0.1 g); initial viability in medium with ox bile (T0 Titer on Ox Bile); viability after 48 hours (48 hr Viable Titer cfu/0.1 g); viability after 48 hours with ox bile (48 hr Titer on Ox Bile); viability after one week (1 week Viable Titer cfu/0.1 g); and viability after one week with ox bile (1 week Titer on Ox Bile). In FIG. 4B, the bars for each of the strains are, from left to right, initial viability (T0 Viable Titer cfu/0.1 g); initial viability in medium with ox bile (T0 Titer on Ox Bile); viability after one month (1 mo Viable Titer cfu/0.1 g); and viability after one month with ox bile (1 mo Titer on Ox Bile).

### DETAILED DESCRIPTION

The preparation and preservation of bacterial compositions for therapeutic use, in particular anaerobic bacteria, has been challenging. While bacteria can be frozen down and regrown on plates or in solution, it has been difficult to standardize this process. There is a need to preserve bacteria that can be used for therapeutic purposes. Lyophilization is a recognized process for the preservation of peptides and proteins. However, lyophilization of bacterial compositions, in particular anaerobic bacteria, has been challenging, and loss of integrity has been found upon the lyophilization of bacterial compositions.

It is relatively straightforward to distinguish between viable and non-viable bacteria. Viable bacteria can divide and cultures can be generated, while non-viable bacteria can no longer be grown up (do not replicate and cannot be cultured). The challenge lies in distinguishing viable bacteria from bacteria that have lost integrity. Bacteria that have lost integrity can still be grown up, meaning the bacterial cells are viable and culturable. However, they will grow more slowly than healthy bacteria.

Additionally, bacteria that have lost integrity may no longer be suitable for use in pharmaceutical compositions. For example, pharmaceutical compositions that transit through the digestive tract when administered to a subject may be exposed to the acidic pH of the stomach. If the active agent(s) of the pharmaceutical composition are intended to reach the intestine and may become exposed to stomach acid, the agent must survive the conditions in the stomach to reach the appropriate site. In addition, if the active agent in the pharmaceutical composition that is targeted for the intestine is a bacterial strain, that bacterial strain must be able to grow in, or at least be able to survive, the environment of the intestine for a sufficient time to be effective.

The methods described herein allow for the assessment of the integrity of bacterial compositions which may be used to produce pharmaceutical compositions, for example as part of a quality control process during the manufacturing of pharmaceutical compositions. If the bacterial compositions are found to lack integrity or have compromised integrity, the bacterial compositions are not used in a pharmaceutical composition. Thus, for instance if pharmaceutical composition comprises one or more bacterial compositions (e.g., if the pharmaceutical composition is a cocktail of bacterial strains), each of those bacterial compositions is tested for integrity. If the integrity of a particular bacterial composition is found to be insufficient, the tested bacterial composition will not be included in the pharmaceutical composition. If a particular bacterial composition is required in a pharmaceutical composition, additional batches of the pharmaceutical composition may be generated and evaluated for integrity. A batch of the particular bacterial composition that has sufficient integrity can then be used to generate the pharmaceutical composition.

Furthermore, pharmaceutical compositions must withstand storage conditions prior to administration to subjects. Bacterial composition lacking integrity or having compromised integrity may not survive storage conditions and therefore, may be less effective upon administration.

It was surprisingly found herein that selective media can be used to distinguish healthy bacterial compositions from bacterial compositions that have lost integrity. As shown herein, both healthy bacterial compositions and bacterial compositions that have lost integrity will grow at a similar rate on non-selective media. Surprisingly, bacterial compositions that have lost integrity were found to have a much slower growth rate on selective media than healthy bacterial compositions. Bacterial compositions as used herein refer to a composition that includes bacteria, such as any of the bacterial strains described herein.

In one aspect, the disclosure provides a method for determining the integrity of a bacterial composition, wherein the method includes a step of growing the bacterial composition on a selective medium. If the bacterial composition grows slower on the selective medium than on a non-selective medium, the integrity of the bacterial composition is compromised. Alternatively, the disclosure provides a method for determining the integrity of a bacterial composition, wherein the method includes a step of growing the bacterial composition on a selective medium. If the bacterial composition grows similarly on the selective medium as on a non-selective medium, the integrity of the bacterial composition is not compromised. The bacterial growth can be measured against a previously obtained standard growth curve for either non-selective or selective media (e.g., a reference growth curve), or the bacterial composition can be grown on both selective medium and non-selective medium in the same evaluations.

In one aspect, the disclosure provides a method for determining the integrity of a first bacterial composition, wherein the method includes: growing the first bacterial composition on a selective medium, growing a second bacterial composition on the selective medium, wherein if the first bacterial composition grows slower than the second bacterial composition, the integrity of the first bacterial composition is compromised. Alternatively, the disclosure provides a method for determining the integrity of a first bacterial composition, wherein the method includes: growing the first bacterial composition on a selective medium, growing a second bacterial composition on the selective medium, wherein if the first bacterial composition grows at as similar rate as the second bacterial composition, the integrity of the first bacterial composition is not compromised. In some embodiments, the first and second bacterial composition include the same bacterial composition but have been prepared differently. Thus, for instance, a first bacterial composition was prepared fresh, while the second bacterial composition is a lyophilized bacterial composition. Generally, the first and second bacterial composition will include the same bacterial strain. However, in some embodiments, related strains (e.g., strains from the same bacterial species) are used in the first and second bacterial composition.

In one aspect, the disclosure provides a method for determining the integrity of a first bacterial composition, wherein the method includes growing the first bacterial composition on a selective medium and on a non-selective medium, growing a second bacterial composition on the selective medium and a non-selective medium, wherein if the difference in growth between the selective medium and the non-selective medium for the first bacterial composition is greater than the difference in growth between the selective medium and the non-selective medium for the second bacterial composition, and both the first bacterial composition and the second bacterial composition grow slower on the selective medium than the non-selective medium, the integrity of the first bacterial composition is compromised. Alternatively, the disclosure provides a method for determining the integrity of a first bacterial composition, wherein the method includes growing the first bacterial composition on a selective medium and on a non-selective medium, growing a second bacterial composition on the selective medium and a non-selective medium, wherein if the difference in growth between the selective medium and the non-selective medium for the first bacterial composition is similar to the difference in growth between the selective medium and the non-selective medium for the second bacterial composition, the integrity of the first bacterial composition is not compromised.

As will be appreciated by one of ordinary skill in the art, the growth of bacteria in a particular medium (e.g., selective, non-selective) may be assessed by any method known in the art. For example, in some embodiments, growth of bacteria in a medium may be assessed by quantifying the colony forming units (cfus). In some embodiments, the growth of bacteria in a medium may be assessed by measuring the optical density of the bacterial culture (e.g., OD₆₀₀), for example over time (e.g., growth curve) or as a maximal density achieved by the culture.

It should be appreciated that the methods disclosed herein can be used as part of quality control. As used herein, the term “quality control” refers to a process or step used to assess the quality of a component, such as a bacterial composition, in the process of producing a product. If the quality of the component meets particular criteria, the component may be used, for example to produce a product; whereas if the quality of the component fails to meet the criteria, the component is not used. In some embodiments, a bacterial composition may be subjected to quality control to assess the bacterial integrity according any of the methods described herein.

In some embodiments, a bacterial composition can undergo one or more steps in a manufacturing process and/or a step to prepare the bacterial compositions for use in a therapeutic (pharmaceutical) composition. Such steps include lyophilization, formulation, drying, equilibration, etc. After the bacterial composition has undergone one or more of the manufacturing/preparation steps, the bacterial composition may be evaluated for bacterial integrity according to the methods provided herein. In some embodiments, a bacterial composition is lyophilized, and the lyophilized bacterial composition is evaluated for loss of integrity. If the bacterial composition is found to have integrity (i.e., was not found to have a loss of integrity), the bacterial composition can be used for further manufacturing and/or preparation steps, for example to produce a pharmaceutical composition. Bacterial compositions found to have integrity (i.e., not found to have a loss of integrity) may be considered to have passed quality control and may be used, for example, in one or more further manufacturing and/or preparation steps (e.g., to produce a product such as a pharmaceutical composition). If the lyophilized bacterial composition is found not to have lost integrity, it is used to produce pharmaceutical compositions. If the bacterial composition is found to have a loss of integrity, the bacterial composition will not be used for further manufacturing and/or preparation steps. Bacterial compositions found to have a loss of integrity may be considered to have failed quality control and are not used, for example, in one or more further manufacturing and/or preparation steps (e.g., not used to produce a product such as a pharmaceutical composition). If the lyophilized bacterial composition is found to have a loss of integrity, it is not used in pharmaceutical compositions.

In some embodiments, bacterial compositions may be subjected to storage conditions for a period of time. In some embodiments, bacterial compositions that have a loss of integrity or have reduced integrity may not remain viable following storage for a period of time. In some embodiments, if a bacterial composition is found to have a loss of integrity, it is not used to product a product that will be subjected to storage conditions.

In some embodiments, the disclosure provides methods of quality control of a pharmaceutical composition comprising multiple bacterial strains. In some embodiments, the methods of quality control comprise growing up each of the bacterial strains of the pharmaceutical compositions separately, followed by lyophilization resulting in a bacterial composition of each of the bacterial strains of the pharmaceutical compositions. Optionally, each of the bacterial compositions may be stored for a prolonged period of time. Each of the bacterial compositions is subsequently tested in one or more of the methods for determining bacterial integrity described herein, and will only be included in the pharmaceutical composition if it has sufficient integrity.

Provided herein are compositions and methods for determining bacterial integrity. Bacterial integrity as used herein generally refers to the health and viability of bacteria and the ability to use the bacteria in manufacturing methods and/or therapeutic compositions (pharmaceutical compositions). In some embodiments, bacteria that do not have sufficient integrity are bacteria that grow slower than healthy bacteria. These bacteria with insufficient integrity, for instance, have a longer lag phase than healthy bacteria. Loss of integrity may be correlated with injury to the bacteria, for instance, the bacteria may have a loss of membrane integrity, loss of cell wall integrity, protein denaturation, cross-linking, chemical modifications, and/or DNA damage. In some embodiments, a bacterial composition is considered to have a loss of integrity or have compromised integrity if the bacterial composition has a slower growth rate as compared to another bacterial composition. In some embodiments, the bacterial composition having a loss of integrity or compromised integrity has a slower growth rate than the same bacterial composition (e.g., same bacterial strain(s)) grown under different conditions (e.g., with non-selective medium). In some embodiments, the bacterial composition having a loss of integrity or compromised integrity has a slower growth rate than another bacterial composition (e.g., containing one or more different bacterial strain(s)) grown under the same conditions (e.g., with selective medium).

In some embodiments, a bacterial composition is considered to have a loss of integrity or have compromised integrity if the bacterial composition has a growth rate (e.g., doubling time) that is longer than the growth rate (e.g., doubling time) of another bacterial composition by at least 5%, 10%, 15%, 20%, 25%, 30%, 35%, 40%, 45%, 50%, 55%, 60%, 65%, 70%, 75% or more. In some embodiments, growth rate of the bacterial composition having a loss of integrity or compromised integrity is slower than the growth rate of the same bacterial composition (e.g., same bacterial strain(s)) grown under different conditions (e.g., with non-selective medium). In some embodiments, the growth rate of the bacterial composition having a loss of integrity or compromised integrity is slower than the growth rate of another bacterial composition (e.g., containing one or more different bacterial strain(s)) grown under the same conditions (e.g., with selective medium).

In some embodiments, a bacterial composition is considered to have a loss of integrity or to have compromised integrity if the bacterial composition has fewer viable bacteria when grown in selective medium, as compared to the bacterial composition grown in non-selective medium. In some embodiments, a bacterial composition is considered to have a loss of integrity or to have compromised integrity if the bacterial composition has fewer viable bacteria when grown in selective medium, as compared to another bacterial composition grown in selective medium. In some embodiments, a bacterial composition is considered to have a loss of integrity or to have compromised integrity if the bacterial composition has a reduction in the viable bacteria by at least 5-fold, 10-fold, 100-fold, 1000-fold, 10⁴-fold, 10⁵-fold or more when grown in selective medium, as compared to the bacterial composition grown in non-selective medium or compared to another bacterial composition grown in selective medium.

In some embodiments of the methods provided herein, the bacterial compositions are grown on selective media. As used herein, the term “selective media” and “selective medium” may be used interchangeably and refer to any growth medium that allows for differentiation between bacterial integrity. In some embodiment, the selective medium contains a selective component, such as a stress-inducing agent. In some embodiments, the stress-inducing agent is bile acid. In some embodiments of the methods provided herein, the selective medium is bile acid media. The methods disclosed herein, include the element of the use of selective media to distinguish between bacterial compositions that have integrity and bacterial compositions that do not have integrity. Selective media are known in the art and allow for the growth of selective microorganisms. As disclosed herein, it was unexpected that selective media can be used to distinguish between bacteria that do or do not have integrity, rather than distinguishing between different bacterial strains, which is the traditional use of selective media.

In general, growth on a bile acid, such as ox bile, measures the viability of the bacterial composition under the stress condition of exposure to bile salts. Bile has antimicrobial properties and can affect the cell membrane, macromolecule stability, and can cause DNA damage in bacterial cells. Assessing bacterial growth in the presence of bile acid relative to the bacterial growth in the absence of bile acid allows for a comparison of bacterial strains under a stress condition that simulates conditions of the human gastrointestinal tract (see, e.g., Begley et al. FEMS Microbiol. Rev (2005) 29:625-651). In some embodiments of the methods provided herein, the selective medium is bile acid media. Bile acids include both primary and secondary bile acids and may be obtained from any source known in the art. Examples of bile acids are taurocholic acid (a derivative of cholic acid), glycocholic acid (a derivative of cholic acid), taurochenodeoxycholic acid (a derivative of chenodeoxycholic acid), and glycochenodeoxycholic acid (a derivative of chenodeoxycholic acid). In some embodiments of the methods provided herein, bile acid media is Ox Bile.

In some embodiments of the methods provided herein, the bacterial compositions are grown on non-selective media. As used herein, the terms “non-selective media” and “non-selective medium” may be used interchangeably. Examples of non-selective medium are known in the art. In some embodiments, the non-selective medium may be the same medium as the selective medium without a selective agent. For example, in some embodiments, the selective medium is chocolate medium with ox bile and the non-selective medium is chocolate medium without ox bile.

As will be appreciated by one of skill in the art, a selective medium and/or a non-selective medium may be in liquid (e.g., broth) or in solid form (e.g., agar).

The methods used herein can be assessed to determine the bacterial integrity of any bacterial composition. In some embodiments, the bacterial composition has undergone one or more manufacturing steps to prepare it for use in a therapeutic composition. In some embodiments, one or more of the bacterial compositions disclosed herein (e.g., the first bacterial composition or the second bacterial composition) was lyophilized prior to being used in the methods provided herein (e.g., prior to being grown on selective or non-selective media). Methods of lyophilizing compositions, including compositions comprising bacteria, are known in the art. See, e.g., U.S. Pat. Nos. 3,261,761; 4,205,132; PCT Publications WO 2014/029578, WO 2012/098358, WO2012/076665 and WO2012/088261, herein incorporated by reference in their entirety.

The methods provided herein can be used to evaluate the bacterial integrity of any bacterial strain. Bacterial strains that can be used in the methods provided herein include aerobic bacteria, anaerobic bacteria including both facultative anaerobes and obligate or strict anaerobes.

In some embodiments of any of the methods provided herein, the first bacterial composition and the second bacterial composition comprise the same bacterial strain.

In some embodiments of any of the methods provided herein, the bacterial strain is an anaerobic bacterial strain. In some embodiments of any of the methods provided herein, the bacterial strain is Clostridium bolteae. In some embodiments of any of the methods provided herein, the bacterial strain is Dorea longicatena.

In some embodiments, the composition includes one or more bacterial strains. In some embodiments of the compositions provided herein, the bacteria are anaerobic bacteria. In some embodiments of the compositions provided herein, the anaerobic bacteria are strict anaerobic bacteria. In some embodiments of the compositions provided herein, the bacteria are from the class Clostridia. In some embodiments of the compositions provided herein, the bacteria are from the family Clostridiaceae. In some embodiments of the compositions provided herein, the bacteria are from the genus Clostridium. In some embodiments of the compositions provided herein, the bacteria belong to Clostridium cluster IV, XIVa, XVI, XVII, or XVIII. In some embodiments of the compositions provided herein, the bacteria belong to Clostridium cluster IV, XIVa, or XVII. In some embodiments of the compositions provided herein, the bacteria belong to Clostridium cluster IV or XIVa.

In some embodiments of the compositions provided herein, the composition includes one or more of the following bacterial strains: Clostridium bolteae, Anaerotruncus colihominis, Eubacterium fissicatena, Clostridium symbiosum, Blautia producta, Dorea longicatena, Erysipelotrichaceae bacterium and Subdolinogranulum spp. In some embodiments of the compositions provided herein, the composition includes one or more of the following bacterial strains: Clostridium bolteae 90A9, Anaerotruncus colihominis DSM17241, Sellimonas intestinalis, Clostridium bacterium UC5.1-1D4, Dorea longicatena CAG:42, Erysipelotrichaceae bacterium 21-3, and Clostridium orbiscindens 1_3_50AFAA. In some embodiments of the compositions provided herein, the composition includes two or more (e.g., 2, 3, 4, 5, 6, 7, or 8) of the following bacterial strains: Clostridium bolteae, Anaerotruncus colihominis, Eubacterium fissicatena, Clostridium symbiosum, Blautia producta, Dorea longicatena, Erysipelotrichaceae bacterium, and Subdolinogranulum spp. In some embodiments, the composition includes Clostridium bolteae. In some embodiments, the composition includes Anaerotruncus colihominis. In some embodiments, the composition includes Eubacterium fissicatena. In some embodiments, the composition includes Clostridium symbiosum. In some embodiments, the composition includes Blautia producta. In some embodiments, the composition includes Dorea longicatena. In some embodiments, the composition includes Erysipelotrichaceae bacterium. In some embodiments, the composition includes Subdolinogranulum spp.

In one aspect, as shown herein (e.g., in the Examples) the methods described herein allow for the determination of bacterial integrity. In one aspect, as shown herein (e.g., in the Examples) the methods provided herein allow for the determination of bacterial integrity of bacterial strains belonging to Clostridium cluster IV, XIVa, or XVII. In one aspect, as shown herein (e.g., in the Examples) the methods provided herein allow for the determination of bacterial integrity of any of the anaerobic bacterial strains Clostridium bolteae, Anaerotruncus colihominis, Eubacterium fissicatena, Clostridium symbiosum, Blautia producta, Dorea longicatena, Erysipelotrichaceae bacterium and/or Subdolinogranulum spp. The exemplary bacterial strains of compositions disclosed herein can also be identified by their 16S rRNA sequences (SEQ ID NOs: 1-8). Identifying bacteria by their sequences furthermore allows for the identification of additional bacterial strains that are identical or highly similar to the exemplified bacteria. For instance, the 16S rRNA sequences of bacterial strains were used to identify the closest relative (based on percent identity) through whole genome sequencing and by comparing these sequences with 16S databases (Table 1). In addition, based on whole genome sequencing and comparing of the whole genome to whole genome databases, the bacterial strains having 16S rRNA sequences provided by SEQ ID NOs: 1-8 are most closely related to the following bacterial species: Clostridium bolteae 90A9, Anaerotruncus colihominis DSM 17241, Dracourtella massiliensis GD1, Clostridium symbiosum WAL-14163, Clostridium bacterium UC5.1-1D4, Dorea longicatena CAG:42, Erysipelotrichaceae bacterium 21_3, and Clostridium orbiscindens 1_3_50AFAA (see, e.g., Table 1). Thus, in one aspect it should be appreciated that each row of Table 1, the bacterial strains are highly similar and/or are identical. In some embodiments, in context of the instant disclosure the names of bacterial strains within a row of Table 1 can be used interchangeably.

TABLE 1
Examples of bacterial species for use in the methods and compositions disclosed herein

| | | | Closest species based on | | | |
|---|---|---|---|---|---|---|
| | SEQ | Closest species based on | Consensus SEQ ID # of 16S | Closest species based on | | |
| Strain | ID | Sanger sequencing of | region as compared with 16S | WGS compared versus | Additional closely | Clostridium |
| number | NO: | 16S region | database | WG databases | related sequences | cluster |
| 1 | 1 | Clostridium bolteae | Clostridium bolteae | Clostridium bolteae 90A9 | | XIVa |
| 2 | 6 | Anaerotruncus | Anaerotruncus colihominis | Anaerotruncus | | IV |
| | | colihominis | | colihominis | | |
| | | | | DSM 17241 | | |
| 3 | 3 | Eubacterium fissicatena | Dracourtella massiliensis | Dracourtella massiliensis | Ruminococcus | XIVa |
| | | | | GD1 | torques; Sellimonas | |
| | | | | | intestinalis | |
| 4 | 4 | Clostridium symbiosum | Clostridium symbiosum | Clostridium symbiosum | | XIVa |
| | | | | WAL-14163 | | |
| 5 | 5 | Blautia producta | Blautia producta | Clostridium bacterium | Blautia producta | XIVa |
| | | | | UC5.1-1D4 | ATCC 27340 | |
| 6 | 2 | Dorea longicatena | Dorea longicatena | Dorea longicatena CAG:42 | | XIVa |
| 7 | 7 | Clostridium innocuum | Clostridium innocuum | Erysipelotrichaceae | | XVIII |
| | | | | bacterium 21_3 | | |
| 8 | 8 | Flavinofractor plautii | Flavinofractor plautii | Clostridium orbiscindens | Subdolinogranulum | IV |
| | | | | 1_3_50AFAA | | |

Aspects of the disclosure relate to bacterial strains with 16S rDNA sequences that have a level of sequence identity to a nucleic acid sequence of any one of the sequences of the bacterial strains or species described herein. Two or more sequences may be assessed for the identity between the sequences. The terms “identical,” percent “identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over an entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the bacterial composition includes one or more bacterial strains, wherein the one or more bacterial strains include one or more 16S rDNA sequences having at least 97% sequence identity with nucleic acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the bacterial composition includes one bacterial strain, wherein the bacterial strain includes one or more 16S rDNA sequences having at least 97% sequence identity with nucleic acid sequences SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some embodiments, the bacterial composition includes one bacterial strain, wherein the bacterial strain includes one or more 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

In some embodiments, the bacterial composition includes one bacterial strain, wherein the bacterial strain includes one or more 16S rDNA sequences having at least 97% sequence identity with the nucleic acid sequence of SEQ ID NO: 1. In some embodiments, the bacterial composition includes one bacterial strain, wherein the bacterial strain includes one or more 16S rDNA sequences having at least 97% sequence identity with the nucleic acid sequence of SEQ ID NO: 2. In some embodiments, the bacterial composition includes one bacterial strain, wherein the bacterial strain includes one or more 16S rDNA sequences having at least 97% sequence identity with the nucleic acid sequence of SEQ ID NO: 3. In some embodiments, the bacterial composition includes one bacterial strain, wherein the bacterial strain includes one or more 16S rDNA sequences having at least 97% sequence identity with the nucleic acid sequence of SEQ ID NO:4. In some embodiments, the bacterial composition includes one bacterial strain, wherein the bacterial strain includes one or more 16S rDNA sequences having at least 97% sequence identity with the nucleic acid sequence of SEQ ID NO:5. In some embodiments, the bacterial composition includes one bacterial strain, wherein the bacterial strain includes one or more 16S rDNA sequences having at least 97% sequence identity with the nucleic acid sequence of SEQ ID NO:6. In some embodiments, the bacterial composition includes one bacterial strain, wherein the bacterial strain includes one or more 16S rDNA sequences having at least 97% sequence identity with the nucleic acid sequence of SEQ ID NO:7. In some embodiments, the bacterial composition includes one bacterial strain, wherein the bacterial strain includes one or more 16S rDNA sequences having at least 97% sequence identity with the nucleic acid sequence of SEQ ID NO:8.

Aspects of the disclosure relate to bacterial strains with 16S rDNA sequences that have homology to a nucleic acid sequence of any one of the sequences of the bacterial strains or species described herein. In some embodiments, the bacterial strain has at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% homology relative to any of the strains or bacterial species described herein over a specified region of nucleic acid or amino acid sequence or over the entire sequence. It would be appreciated by one of skill in the art that the term “homology” or “percent homology,” in the context of two or more nucleic acid sequences or amino acid sequences, refers to a measure of similarity between two or more sequences or portion(s) thereof. The homology may exist over a region of a sequence that is at least about 50 nucleotides in length, or more preferably over a region that is 100 to 500 or 1000 or more nucleotides in length. In some embodiments, the homology exists over the length the 16S rRNA or 16S rDNA sequence, or a portion thereof.

In some embodiments, the bacterial composition includes one or more bacterial strains, wherein the one or more bacterial strains include one or more 16S rDNA sequences having at least 97% homology with nucleic acid sequences of SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the bacterial composition includes one bacterial strain, wherein the bacterial strain includes one or more 16S rDNA sequences having at least 97% homology with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8. In some embodiments, the bacterial composition includes one bacterial strain, wherein the bacterial strain includes one or more 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% homology with nucleic acid sequences SEQ ID NO:1, SEQ ID NO:2, SEQ ID NO:3, SEQ ID NO:4, SEQ ID NO:5, SEQ ID NO:6, SEQ ID NO:7, or SEQ ID NO:8.

In some embodiments, the bacterial composition includes one bacterial strain, wherein the bacterial strain includes one or more 16S rDNA sequences having at least 97% homology with the nucleic acid sequence of SEQ ID NO:1. In some embodiments, the bacterial composition includes one bacterial strain, wherein the bacterial strain includes one or more 16S rDNA sequences having at least 97% homology with the nucleic acid sequence of SEQ ID NO:2. In some embodiments, the bacterial composition includes one bacterial strain, wherein the bacterial strain includes one or more 16S rDNA sequences having at least 97% homology with the nucleic acid sequence of SEQ ID NO:3. In some embodiments, the bacterial composition includes one bacterial strain, wherein the bacterial strain includes one or more 16S rDNA sequences having at least 97% homology with the nucleic acid sequence of SEQ ID NO:4. In some embodiments, the bacterial composition includes one bacterial strain, wherein the bacterial strain includes one or more 16S rDNA sequences having at least 97% homology with the nucleic acid sequence of SEQ ID NO:5. In some embodiments, the bacterial composition includes one bacterial strain, wherein the bacterial strain includes one or more 16S rDNA sequences having at least 97% homology with the nucleic acid sequence of SEQ ID NO:6. In some embodiments, the bacterial composition includes one bacterial strain, wherein the bacterial strain includes one or more 16S rDNA sequences having at least 97% homology with the nucleic acid sequence of SEQ ID NO:7. In some embodiments, the bacterial composition includes one bacterial strain, wherein the bacterial strain includes one or more 16S rDNA sequences having at least 97% homology with the nucleic acid sequence of SEQ ID NO:8.

In some embodiments, the composition includes a bacterial strain that includes a 16S RNA nucleic acid sequence with at least 97% sequence identity with the 16S RNA sequence of SEQ ID NO:1. In some embodiments, the composition includes a bacterial strain that includes a 16S RNA nucleic acid sequence with at least 97% sequence identity with the 16S RNA sequence of SEQ ID NO:2.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region of the nucleic acid or amino acid sequence or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, by computerized implementations of these algorithms (GAP, BESTFIT, FASTA, and TFASTA in the Wisconsin Genetics Software Package, Genetics Computer Group. Madison. Wis.), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402, and Altschul et al., J. Mol. Biol. (1990) 215:403-410, respectively.

It should be appreciated that the terms bacteria and bacterial strains as used herein are interchangeable.

In some embodiments, the bacterial compositions disclosed herein can be used in therapeutic applications. In some embodiments, the bacterial compositions disclosed herein can be used in pharmaceutical compositions. In some embodiments, the bacterial compositions used in therapeutic applications and/or pharmaceutical compositions, are bacterial compositions that were found to have integrity based on the methods provided herein (i.e., were not found to have a loss of integrity). In some embodiments, the solid compositions that include bacterial strains provided herein may be formulated for administration as a pharmaceutical composition, e.g., by reconstitution of a lyophilized product. It should be appreciated that a second aliquot of the bacterial compositions disclosed herein may be used in a pharmaceutical composition. Thus, in some embodiments a first aliquot is used in the methods provided herein to evaluate the integrity of the bacterial composition. If the first aliquot of the bacterial compositions was found to have integrity based on the methods provided herein (i.e., was not found to have a loss of integrity), a second aliquot or additional aliquots of the bacterial composition may be used in a pharmaceutical composition.

The term “pharmaceutical composition” as used herein means a product that results from the mixing or combining of a solid formulation provided herein and one or more pharmaceutically acceptable excipient.

An “acceptable” excipient refers to an excipient that must be compatible with the active ingredient (e.g., the bacterial strain) and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Strain 1 16S ribosomal RNA Clostridium bolteae
(SEQ ID NO: 1)
ATGAGAGTTTGATCCTGGCTCAGGATGAACGCTGGCGGCGTGCCTAACACATGCAAGTCGAACGAAGC
AATTAAAATGAAGTTTTCGGATGGATTTTTGATTGACTGAGTGGCGGACGGGTGAGTAACGCGTGGAT
AACCTGCCTCACACTGGGGGATAACAGTTAGAAATGACTGCTAATACCGCATAAGCGCACAGTACCGC
ATGGTACGGTGTGAAAAACTCCGGTGGTGTGAGATGGATCCGCGTCTGATTAGCCAGTTGGCGGGGTA
ACGGCCCACCAAAGCGACGATCAGTAGCCGACCTGAGAGGGTGACCGGCCACATTGGGACTGAGACAC
GGCCCAAACTCCTACGGGAGGCAGCAGTGGGGAATATTGCACAATGGGCGAAAGCCTGATGCAGCGAC
GCCGCGTGAGTGAAGAAGTATTTCGGTATGTAAAGCTCTATCAGCAGGGAAGAAAATGACGGTACCTG
ACTAAGAAGCCCCGGCTAACTACGTGCCAGCAGCCGCGGTAATACGTAGGGGGCAAGCGTTATCCGGA
TTTACTGGGTGTAAAGGGAGCGTAGACGGCGAAGCAAGTCTGAAGTGAAAACCCAGGGCTCAACCCTG
GGACTGCTTTGGAAACTGTTTTGCTAGAGTGTCGGAGAGGTAAGTGGAATTCCTAGTGTAGCGGTGAA
ATGCGTAGATATTAGGAGGAACACCAGTGGCGAAGGCGGCTTACTGGACGATAACTGACGTTGAGGCT
CGAAAGCGTGGGGAGCAAACAGGATTAGATACCCTGGTAGTCCACGCCGTAAACGATGAATGCTAGGT
GTTGGGGGGCAAAGCCCTTCGGTGCCGTCGCAAACGCAGTAAGCATTCCACCTGGGGAGTACGTTCGC
AAGAATGAAACTCAAAGGAATTGACGGGGACCCGCACAAGCGGTGGAGCATGTGGTTTAATTCGAAGC
AACGCGAAGAACCTTACCAAGTCTTGACATCCTCTTGACCGGCGTGTAACGGCGCCTTCCCTTCGGGG
CAAGAGAGACAGGTGGTGCATGGTTGTCGTCAGCTCGTGTCGTGAGATGTTGGGTTAAGTCCCGCAAC
GAGCGCAACCCTTATCCTTAGTAGCCAGCAGGTAAAGCTGGGCACTCTAGGGAGACTGCCAGGGATAA
CCTGGAGGAAGGTGGGGATGACGTCAAATCATCATGCCCCTTATGATTTGGGCTACACACGTGCTACA
ATGGCGTAAACAAAGGGAAGCAAGACAGTGATGTGGAGCAAATCCCAAAAATAACGTCCCAGTTCGGA
CTGTAGTCTGCAACCCGACTACACGAAGCTGGAATCGCTAGTAATCGCGAATCAGAATGTCGCGGTGA
ATACGTTCCCGGGTCTTGTACACACCGCCCGTCACACCATGGGAGTCAGCAACGCCCGAAGTCAGTGA
CCCAACTCGCAAGAGAGGGAGCTGCCGAAGGCGGGGCAGGTAACTGGGGTGAAGTCGTAACAAGGTAG
CCGTATCGGAAGGTGCGGCTGGATCACCTCCTTT
Strain 2 16S ribosomal RNA Anaerotruncus colihominis
(SEQ ID NO: 6)
TCAAAGAGTTTGATCCTGGCTCAGGACGAACGCTGGCGGCGCGCCTAACACATGCAAGTCGAACGGAG
CTTACGTTTTGAAGTTTTCGGATGGATGAATGTAAGCTTAGTGGCGGACGGGTGAGTAACACGTGAGC
AACCTGCCTTTCAGAGGGGGATAACAGCCGGAAACGGCTGCTAATACCGCATGATGTTGCGGGGGCAC
ATGCCCCTGCAACCAAAGGAGCAATCCGCTGAAAGATGGGCTCGCGTCCGATTAGCCAGTTGGCGGGG
TAACGGCCCACCAAAGCGACGATCGGTAGCCGGACTGAGAGGTTGAACGGCCACATTGGGACTGAGAC
ACGGCCCAGACTCCTACGGGAGGCAGCAGTGGGGGATATTGCACAATGGGCGAAAGCCTGATGCAGCG
ACGCCGCGTGAGGGAAGACGGTCTTCGGATTGTAAACCTCTGTCTTTGGGGAAGAAAATGACGGTACC
CAAAGAGGAAGCTCCGGCTAACTACGTGCCAGCAGCCGCGGTAATACGTAGGGAGCAAGCGTTGTCCG
GAATTACTGGGTGTAAAGGGAGCGTAGGCGGGATGGCAAGTAGAATGTTAAATCCATCGGCTCAACCG
GTGGCTGCGTTCTAAACTGCCGTTCTTGAGTGAAGTAGAGGCAGGCGGAATTCCTAGTGTAGCGGTGA
AATGCGTAGATATTAGGAGGAACACCAGTGGCGAAGGCGGCCTGCTGGGCTTTAACTGACGCTGAGGC
TCGAAAGCGTGGGGAGCAAACAGGATTAGATACCCTGGTAGTCCACGCCGTAAACGATGATTACTAGG
TGTGGGGGGACTGACCCCTTCCGTGCCGCAGTTAACACAATAAGTAATCCACCTGGGGAGTACGGCCG
CAAGGTTGAAACTCAAAGGAATTGACGGGGGCCCGCACAAGCAGTGGAGTATGTGGTTTAATTCGAAG
CAACGCGAAGAACCTTACCAGGTCTTGACATCGGATGCATAGCCTAGAGATAGGTGAAGCCCTTCGGG
GCATCCAGACAGGTGGTGCATGGTTGTCGTCAGCTCGTGTCGTGAGATGTTGGGTTAAGTCCCGCAAC
GAGCGCAACCCTTATTATTAGTTGCTACGCAAGAGCACTCTAATGAGACTGCCGTTGACAAAACGGAG
GAAGGTGGGGATGACGTCAAATCATCATGCCCCTTATGACCTGGGCTACACACGTACTACAATGGCAC
TAAAACAGAGGGCGGCGACACCGCGAGGTGAAGCGAATCCCGAAAAAGTGTCTCAGTTCAGATTGCAG
GCTGCAACCCGCCTGCATGAAGTCGGAATTGCTAGTAATCGCGGATCAGCATGCCGCGGTGAATACGT
TCCCGGGCCTTGTACACACCGCCCGTCACACCATGGGAGTCGGTAACACCCGAAGCCAGTAGCCTAAC
CGCAAGGGGGGCGCTGTCGAAGGTGGGATTGATGACTGGGGTGAAGTCGTAACAAGGTAGCCGTATCG
GAAGGTGCGGCTGGATCACCTCCTTT
Strain 3 16S ribosomal RNA Ruminococcus torques
(SEQ ID NO: 3)
TACGAGAGTTTGATCCTGGCTCAGGATGAACGCTGGCGGCGTGCCTAACACATGCAAGTCGAGCGAAG
CGCTGTTTTCAGAATCTTCGGAGGAAGAGGACAGTGACTGAGCGGCGGACGGGTGAGTAACGCGTGGG
CAACCTGCCTCATACAGGGGGATAACAGTTAGAAATGACTGCTAATACCGCATAAGCGCACAGGACCG
CATGGTGTAGTGTGAAAAACTCCGGTGGTATGAGATGGACCCGCGTCTGATTAGGTAGTTGGTGGGGT
AAAGGCCTACCAAGCCGACGATCAGTAGCCGACCTGAGAGGGTGACCGGCCACATTGGGACTGAGACA
CGGCCCAAACTCCTACGGGAGGCAGCAGTGGGGAATATTGCACAATGGGGGAAACCCTGATGCAGCGA
CGCCGCGTGAAGGAAGAAGTATTTCGGTATGTAAACTTCTATCAGCAGGGAAGAAAATGACGGTACCT
GAGTAAGAAGCACCGGCTAAATACGTGCCAGCAGCCGCGGTAATACGTATGGTGCAAGCGTTATCCGG
ATTTACTGGGTGTAAAGGGAGCGTAGACGGATAGGCAAGTCTGGAGTGAAAACCCAGGGCTCAACCCT
GGGACTGCTTTGGAAACTGCAGATCTGGAGTGCCGGAGAGGTAAGCGGAATTCCTAGTGTAGCGGTGA
AATGCGTAGATATTAGGAGGAACACCAGTGGCGAAGGCGGCTTACTGGACGGTGACTGACGTTGAGGC
TCGAAAGCGTGGGGAGCAAACAGGATTAGATACCCTGGTAGTCCACGCCGTAAACGATGACTACTAGG
TGTCGGTGTGCAAAGCACATCGGTGCCGCAGCAAACGCAATAAGTAGTCCACCTGGGGAGTACGTTCG
CAAGAATGAAACTCAAAGGAATTGACGGGGACCCGCACAAGCGGTGGAGCATGTGGTTTAATTCGAAG
CAACGCGAAGAACCTTACCTGGTCTTGACATCCGGATGACGGGCGAGTAATGTCGCCGTCCCTTCGGG
GCGTCCGAGACAGGTGGTGCATGGTTGTCGTCAGCTCGTGTCGTGAGATGTTGGGTTAAGTCCCGCAA
CGAGCGCAACCCTTATCTTCAGTAGCCAGCATATAAGGTGGGCACTCTGGAGAGACTGCCAGGGAGAA
CCTGGAGGAAGGTGGGGATGACGTCAAATCATCATGCCCCTTATGGCCAGGGCTACACACGTGCTACA
ATGGCGTAAACAAAGGGAAGCGAGAGGGTGACCTGGAGCGAATCCCAAAAATAACGTCTCAGTTCGGA
TTGTAGTCTGCAACTCGACTACATGAAGCTGGAATCGCTAGTAATCGCGGATCAGCATGCCGCGGTGA
ATACGTTCCCGGGTCTTGTACACACCGCCCGTCACACCATGGGAGTCAGTAACGCCCGAAGCCAGTGA
CCCAACCTTAGAGGAGGGAGCTGTCGAAGGCGGGACGGATAACTGGGGTGAAGTCGTAACAAGGTAGC
CGTATCGGAAGGTGCGGCTGGATCACCTCCTTT
Strain 4 16S ribosomal RNA Clostridium symbiosum
(SEQ ID NO: 4)
ATGAGAGTTTGATCCTGGCTCAGGATGAACGCTGGCGGCGTGCCTAACACATGCAAGTCGAACGAAGC
GATTTAACGGAAGTTTTCGGATGGAAGTTGAATTGACTGAGTGGCGGACGGGTGAGTAACGCGTGGGT
AACCTGCCTTGTACTGGGGGACAACAGTTAGAAATGACTGCTAATACCGCATAAGCGCACAGTATCGC
ATGATACAGTGTGAAAAACTCCGGTGGTACAAGATGGACCCGCGTCTGATTAGCTAGTTGGTAAGGTA
ACGGCTTACCAAGGCGACGATCAGTAGCCGACCTGAGAGGGTGACCGGCCACATTGGGACTGAGACAC
GGCCCAAACTCCTACGGGAGGCAGCAGTGGGGAATATTGCACAATGGGCGAAAGCCTGATGCAGCGAC
GCCGCGTGAGTGAAGAAGTATTTCGGTATGTAAAGCTCTATCAGCAGGGAAGAAAATGACGGTACCTG
ACTAAGAAGCCCCGGCTAACTACGTGCCAGCAGCCGCGGTAATACGTAGGGGGCAAGCGTTATCCGGA
TTTACTGGGTGTAAAGGGAGCGTAGACGGTAAAGCAAGTCTGAAGTGAAAGCCCGCGGCTCAACTGCG
GGACTGCTTTGGAAACTGTTTAACTGGAGTGTCGGAGAGGTAAGTGGAATTCCTAGTGTAGCGGTGAA
ATGCGTAGATATTAGGAGGAACACCAGTGGCGAAGGCGACTTACTGGACGATAACTGACGTTGAGGCT
CGAAAGCGTGGGGAGCAAACAGGATTAGATACCCTGGTAGTCCACGCCGTAAACGATGAATACTAGGT
GTTGGGGAGCAAAGCTCTTCGGTGCCGTCGCAAACGCAGTAAGTATTCCACCTGGGGAGTACGTTCGC
AAGAATGAAACTCAAAGGAATTGACGGGGACCCGCACAAGCGGTGGAGCATGTGGTTTAATTCGAAGC
AACGCGAAGAACCTTACCAGGTCTTGACATCGATCCGACGGGGGAGTAACGTCCCCTTCCCTTCGGGG
CGGAGAAGACAGGTGGTGCATGGTTGTCGTCAGCTCGTGTCGTGAGATGTTGGGTTAAGTCCCGCAAC
GAGCGCAACCCTTATTCTAAGTAGCCAGCGGTTCGGCCGGGAACTCTTGGGAGACTGCCAGGGATAAC
CTGGAGGAAGGTGGGGATGACGTCAAATCATCATGCCCCTTATGATCTGGGCTACACACGTGCTACAA
TGGCGTAAACAAAGAGAAGCAAGACCGCGAGGTGGAGCAAATCTCAAAAATAACGTCTCAGTTCGGAC
TGCAGGCTGCAACTCGCCTGCACGAAGCTGGAATCGCTAGTAATCGCGAATCAGAATGTCGCGGTGAA
TACGTTCCCGGGTCTTGTACACACCGCCCGTCACACCATGGGAGTCAGTAACGCCCGAAGTCAGTGAC
CCAACCGCAAGGAGGGAGCTGCCGAAGGCGGGACCGATAACTGGGGTGAAGTCGTAACAAGGTAGCCG
TATCGGAAGGTGCGGCTGGATCACCTCCTTT
Strain 5 16S ribosomal RNA Blautia producta
(SEQ ID NO: 5)
ATCAGAGAGTTTGATCCTGGCTCAGGATGAACGCTGGCGGCGTGCTTAACACATGCAAGTCGAGCGAA
GCACTTAAGTGGATCTCTTCGGATTGAAGCTTATTTGACTGAGCGGCGGACGGGTGAGTAACGCGTGG
GTAACCTGCCTCATACAGGGGGATAACAGTTAGAAATGGCTGCTAATACCGCATAAGCGCACAGGACC
GCATGGTCTGGTGTGAAAAACTCCGGTGGTATGAGATGGACCCGCGTCTGATTAGCTAGTTGGAGGGG
TAACGGCCCACCAAGGCGACGATCAGTAGCCGGCCTGAGAGGGTGAACGGCCACATTGGGACTGAGAC
ACGGCCCAGACTCCTACGGGAGGCAGCAGTGGGGAATATTGCACAATGGGGGAAACCCTGATGCAGCG
ACGCCGCGTGAAGGAAGAAGTATCTCGGTATGTAAACTTCTATCAGCAGGGAAGAAAATGACGGTACC
TGACTAAGAAGCCCCGGCTAACTACGTGCCAGCAGCCGCGGTAATACGTAGGGGGCAAGCGTTATCCG
GATTTACTGGGTGTAAAGGGAGCGTAGACGGAAGAGCAAGTCTGATGTGAAAGGCTGGGGCTTAACCC
CAGGACTGCATTGGAAACTGTTTTTCTAGAGTGCCGGAGAGGTAAGCGGAATTCCTAGTGTAGCGGTG
AAATGCGTAGATATTAGGAGGAACACCAGTGGCGAAGGCGGCTTACTGGACGGTAACTGACGTTGAGG
CTCGAAAGCGTGGGGAGCAAACAGGATTAGATACCCTGGTAGTCCACGCCGTAAACGATGAATACTAG
GTGTCGGGTGGCAAAGCCATTCGGTGCCGCAGCAAACGCAATAAGTATTCCACCTGGGGAGTACGTTC
GCAAGAATGAAACTCAAAGGAATTGACGGGGACCCGCACAAGCGGTGGAGCATGTGGTTTAATTCGAA
GCAACGCGAAGAACCTTACCAAGTCTTGACATCCCTCTGACCGGCCCGTAACGGGGCCTTCCCTTCGG
GGCAGAGGAGACAGGTGGTGCATGGTTGTCGTCAGCTCGTGTCGTGAGATGTTGGGTTAAGTCCCGCA
ACGAGCGCAACCCCTATCCTTAGTAGCCAGCAGGTGAAGCTGGGCACTCTAGGGAGACTGCCGGGGAT
AACCCGGAGGAAGGCGGGGACGACGTCAAATCATCATGCCCCTTATGATTTGGGCTACACACGTGCTA
CAATGGCGTAAACAAAGGGAAGCGAGACAGCGATGTTGAGCAAATCCCAAAAATAACGTCCCAGTTCG
GACTGCAGTCTGCAACTCGACTGCACGAAGCTGGAATCGCTAGTAATCGCGAATCAGAATGTCGCGGT
GAATACGTTCCCGGGTCTTGTACACACCGCCCGTCACACCATGGGAGTCAGTAACGCCCGAAGTCAGT
GACCCAACCTTACAGGAGGGAGCTGCCGAAGGCGGGACCGATAACTGGGGTGAAGTCGTAACAAGGTA
GCCGTATCGGAAGGTGCGGCTGGATCACCTCCTTT
Strain 6 16S ribosomal RNA Dorea longicatena
(SEQ ID NO: 2)
AACGAGAGTTTGATCCTGGCTCAGGATGAACGCTGGCGGCGTGCTTAACACATGCAAGTCGAGCGAAG
CACTTAAGTTTGATTCTTCGGATGAAGACTTTTGTGACTGAGCGGCGGACGGGTGAGTAACGCGTGGG
TAACCTGCCTCATACAGGGGGATAACAGTTAGAAATGACTGCTAATACCGCATAAGACCACGGTACCG
CATGGTACAGTGGTAAAAACTCCGGTGGTATGAGATGGACCCGCGTCTGATTAGGTAGTTGGTGGGGT
AACGGCCTACCAAGCCGACGATCAGTAGCCGACCTGAGAGGGTGACCGGCCACATTGGGACTGAGACA
CGGCCCAGACTCCTACGGGAGGCAGCAGTGGGGAATATTGCACAATGGAGGAAACTCTGATGCAGCGA
CGCCGCGTGAAGGATGAAGTATTTCGGTATGTAAACTTCTATCAGCAGGGAAGAAAATGACGGTACCT
GACTAAGAAGCCCCGGCTAACTACGTGCCAGCAGCCGCGGTAATACGTAGGGGGCAAGCGTTATCCGG
ATTTACTGGGTGTAAAGGGAGCGTAGACGGCACGGCAAGCCAGATGTGAAAGCCCGGGGCTCAACCCC
GGGACTGCATTTGGAACTGCTGAGCTAGAGTGTCGGAGAGGCAAGTGGAATTCCTAGTGTAGCGGTGA
AATGCGTAGATATTAGGAGGAACACCAGTGGCGAAGGCGGCTTGCTGGACGATGACTGACGTTGAGGC
TCGAAAGCGTGGGGAGCAAACAGGATTAGATACCCTGGTAGTCCACGCCGTAAACGATGACTGCTAGG
TGTCGGGTGGCAAAGCCATTCGGTGCCGCAGCTAACGCAATAAGCAGTCCACCTGGGGAGTACGTTCG
CAAGAATGAAACTCAAAGGAATTGACGGGGACCCGCACAAGCGGTGGAGCATGTGGTTTAATTCGAAG
CAACGCGAAGAACCTTACCTGATCTTGACATCCCGATGACCGCTTCGTAATGGAAGCTTTTCTTCGGA
ACATCGGTGACAGGTGGTGCATGGTTGTCGTCAGCTCGTGTCGTGAGATGTTGGGTTAAGTCCCGCAA
CGAGCGCAACCCCTATCTTCAGTAGCCAGCAGGTTAAGCTGGGCACTCTGGAGAGACTGCCAGGGATA
ACCTGGAGGAAGGTGGGGATGACGTCAAATCATCATGCCCCTTATGACCAGGGCTACACACGTGCTAC
AATGGCGTAAACAAAGAGAAGCGAACTCGCGAGGGTAAGCAAATCTCAAAAATAACGTCTCAGTTCGG
ATTGTAGTCTGCAACTCGACTACATGAAGCTGGAATCGCTAGTAATCGCAGATCAGAATGCTGCGGTG
AATACGTTCCCGGGTCTTGTACACACCGCCCGTCACACCATGGGAGTCAGTAACGCCCGAAGTCAGTG
ACCCAACCGTAAGGAGGGAGCTGCCGAAGGTGGGACCGATAACTGGGGTGAAGTCGTAACAAGGTAGC
CGTATCGGAAGGTGCGGCTGGATCACCTCCTTT
Strain 7 16S ribosomal RNA Erysipelotrichaceae bacterium
(SEQ ID NO: 7)
ATGGAGAGTTTGATCCTGGCTCAGGATGAACGCTGGCGGCATGCCTAATACATGCAAGTCGAACGAAG
TTTCGAGGAAGCTTGCTTCCAAAGAGACTTAGTGGCGAACGGGTGAGTAACACGTAGGTAACCTGCCC
ATGTGTCCGGGATAACTGCTGGAAACGGTAGCTAAAACCGGATAGGTATACAGAGCGCATGCTCAGTA
TATTAAAGCGCCCATCAAGGCGTGAACATGGATGGACCTGCGGCGCATTAGCTAGTTGGTGAGGTAAC
GGCCCACCAAGGCGATGATGCGTAGCCGGCCTGAGAGGGTAAACGGCCACATTGGGACTGAGACACGG
CCCAAACTCCTACGGGAGGCAGCAGTAGGGAATTTTCGTCAATGGGGGAAACCCTGAACGAGCAATGC
CGCGTGAGTGAAGAAGGTCTTCGGATCGTAAAGCTCTGTTGTAAGTGAAGAACGGCTCATAGAGGAAA
TGCTATGGGAGTGACGGTAGCTTACCAGAAAGCCACGGCTAACTACGTGCCAGCAGCCGCGGTAATAC
GTAGGTGGCAAGCGTTATCCGGAATCATTGGGCGTAAAGGGTGCGTAGGTGGCGTACTAAGTCTGTAG
TAAAAGGCAATGGCTCAACCATTGTAAGCTATGGAAACTGGTATGCTGGAGTGCAGAAGAGGGCGATG
GAATTCCATGTGTAGCGGTAAAATGCGTAGATATATGGAGGAACACCAGTGGCGAAGGCGGTCGCCTG
GTCTGTAACTGACACTGAGGCACGAAAGCGTGGGGAGCAAATAGGATTAGATACCCTAGTAGTCCACG
CCGTAAACGATGAGAACTAAGTGTTGGAGGAATTCAGTGCTGCAGTTAACGCAATAAGTTCTCCGCCT
GGGGAGTATGCACGCAAGTGTGAAACTCAAAGGAATTGACGGGGGCCCGCACAAGCGGTGGAGTATGT
GGTTTAATTCGAAGCAACGCGAAGAACCTTACCAGGCCTTGACATGGAAACAAATACCCTAGAGATAG
GGGGATAATTATGGATCACACAGGTGGTGCATGGTTGTCGTCAGCTCGTGTCGTGAGATGTTGGGTTA
AGTCCCGCAACGAGCGCAACCCTTGTCGCATGTTACCAGCATCAAGTTGGGGACTCATGCGAGACTGC
CGGTGACAAACCGGAGGAAGGTGGGGATGACGTCAAATCATCATGCCCCTTATGGCCTGGGCTACACA
CGTACTACAATGGCGGCCACAAAGAGCAGCGACACAGTGATGTGAAGCGAATCTCATAAAGGTCGTCT
CAGTTCGGATTGAAGTCTGCAACTCGACTTCATGAAGTCGGAATCGCTAGTAATCGCAGATCAGCATG
CTGCGGTGAATACGTTCTCGGGCCTTGTACACACCGCCCGTCAAACCATGGGAGTCAGTAATACCCGA
AGCCGGTGGCATAACCGTAAGGAGTGAGCCGTCGAAGGTAGGACCGATGACTGGGGTTAAGTCGTAAC
AAGGTATCCCTACGGGAACGTGGGGATGGATCACCTCCTTT
Strain 8 16S ribosomal RNA Subdoligranulum spp
(SEQ ID NO: 8)
TATTGAGAGTTTGATCCTGGCTCAGGATGAACGCTGGCGGCGTGCTTAACACATGCAAGTCGAACGGG
GTGCTCATGACGGAGGATTCGTCCAACGGATTGAGTTACCTAGTGGCGGACGGGTGAGTAACGCGTGA
GGAACCTGCCTTGGAGAGGGGAATAACACTCCGAAAGGAGTGCTAATACCGCATGATGCAGTTGGGTC
GCATGGCTCTGACTGCCAAAGATTTATCGCTCTGAGATGGCCTCGCGTCTGATTAGCTAGTAGGCGGG
GTAACGGCCCACCTAGGCGACGATCAGTAGCCGGACTGAGAGGTTGACCGGCCACATTGGGACTGAGA
CACGGCCCAGACTCCTACGGGAGGCAGCAGTGGGGAATATTGGGCAATGGGCGCAAGCCTGACCCAGC
AACGCCGCGTGAAGGAAGAAGGCTTTCGGGTTGTAAACTTCTTTTGTCGGGGACGAAACAAATGACGG
TACCCGACGAATAAGCCACGGCTAACTACGTGCCAGCAGCCGCGGTAATACGTAGGTGGCAAGCGTTA
TCCGGATTTACTGGGTGTAAAGGGCGTGTAGGCGGGATTGCAAGTCAGATGTGAAAACTGGGGGCTCA
ACCTCCAGCCTGCATTTGAAACTGTAGTTCTTGAGTGCTGGAGAGGCAATCGGAATTCCGTGTGTAGC
GGTGAAATGCGTAGATATACGGAGGAACACCAGTGGCGAAGGCGGATTGCTGGACAGTAACTGACGCT
GAGGCGCGAAAGCGTGGGGAGCAAACAGGATTAGATACCCTGGTAGTCCACGCCGTAAACGATGGATA
CTAGGTGTGGGGGGTCTGACCCCCTCCGTGCCGCAGTTAACACAATAAGTATCCCACCTGGGGAGTAC
GATCGCAAGGTTGAAACTCAAAGGAATTGACGGGGGCCCGCACAAGCGGTGGAGTATGTGGTTTAATT
CGAAGCAACGCGAAGAACCTTACCAGGGCTTGACATCCCACTAACGAAGCAGAGATGCATTAGGTGCC
CTTCGGGGAAAGTGGAGACAGGTGGTGCATGGTTGTCGTCAGCTCGTGTCGTGAGATGTTGGGTTAAG
TCCCGCAACGAGCGCAACCCTTATTGTTAGTTGCTACGCAAGAGCACTCTAGCGAGACTGCCGTTGAC
AAAACGGAGGAAGGTGGGGACGACGTCAAATCATCATGCCCCTTATGTCCTGGGCCACACACGTACTA
CAATGGTGGTTAACAGAGGGAGGCAATACCGCGAGGTGGAGCAAATCCCTAAAAGCCATCCCAGTTCG
GATTGCAGGCTGAAACCCGCCTGTATGAAGTTGGAATCGCTAGTAATCGCGGATCAGCATGCCGCGGT
GAATACGTTCCCGGGCCTTGTACACACCGCCCGTCACACCATGAGAGTCGGGAACACCCGAAGTCCGT
AGCCTAACCGCAAGGAGGGCGCGGCCGAAGGTGGGTTCGATAATTGGGGTGAAGTCGTAACAAGGTAG
CCGTATCGGAAGGTGCGGCTGGATCACCTCCTTT

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Evaluating Bacterial Integrity Using Selective Media

Experiment 1: Dorea longicatena

A lyophilized culture of Dorea longicatena (Strain 6, with 16S RNA Sequence SEQ ID NO:2) was prepared by growing in HiVeg reinforced clostridial media until early stage stationary phase, and then exchanging with 8% sucrose, 1% yeast extract, 0.05% cysteine, 20 mM histidine, pH7 via diafiltration. This was frozen at 1° C. per minute followed by lyophilization. The overall titer after lyophilization was reduced by approximately 3 logs from the starting culture. The lyophilized culture was reconstituted in PBS before use.

A frozen culture of Dorea longicatena was created by mixing 6 ml of a late log phase culture in PYG with 2 ml of 60% glycerol to a final concentration of 15% glycerol. This was frozen at −80° C. for at least 1 week. The culture was thawed at room temperature before use.

A healthy fresh culture of Dorea longicatena was created by inoculating a fresh colony into reduced PYG media and incubating at 37° C. in anaerobic conditions overnight.

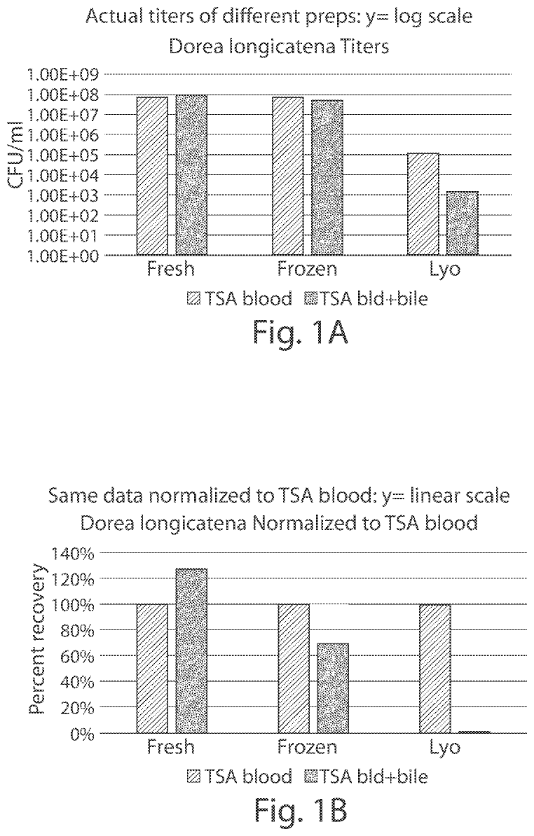

Each of the above Dorea longicatena cultures were serially diluted in PBS. 100 ul of dilutions were plated in duplicate on pre-reduced Trypticase Soy Agar supplemented with 5% horse blood (TSBA) and pre-reduced TSAB with the addition of 0.15% Ox Bile (the bile acid media). Petri plates were incubated anaerobically for 3 days at 37° C. and then the colonies were counted and the titers calculated based on the dilutions and plated volume. The results are shown in FIGS. 1A and 1B.

The PYG control data were generated using a BioscreenC assay: 50 ul of dilutions were added to 350 ul reduced PYG in wells of a BioscreenC growth curve analyzer. For the fresh culture, four replicates of the 10⁻² and 10⁻⁴ dilutions, five replicates of the 10⁻⁵ and 10⁻⁶, twenty replicates of 10⁻⁷, thirty replicates of 10⁻⁸ and thirty eight replicates of 10⁻⁹ were used. No growth was observed in the 10⁻⁹ dilution. For the lyophilized culture, four replicates of the 10⁻² and 10⁻⁴ dilutions, ten replicates of 10⁻⁵, thirty replicates of 10⁻⁶, and forty replicates of 10⁻⁷ were used.

Using the lowest three dilutions that showed growth (10⁻⁶, 10⁻⁷, and 10⁻⁸ for fresh, and 10⁻⁵, 10⁻⁶, and 10⁻⁷ for lyophilized), the titer was estimated using a most probable number calculation, solving for X iteratively from the following equation:

$${\sum }_{j = 1}^{k} \frac{{g}_{j} {m}_{j}}{1 - exp ( - λ {m}_{j} )} = {\sum }_{j = 1}^{k} {t}_{j} {m}_{j}$$

where exp(x) means e′, and

K denotes the number of dilutions,

gⱼ denotes the number of positive (or growth) weds in the jth dilution,

mⱼ denotes the amount of the original sample put in each well in the jth dilution,

tⱼ denotes the number of we in the jth dilution.

The results are shown in FIGS. 1A and 1B.

Experiment 2: Clostridium bolteae

A lyophilized culture of Clostridium bolteae (Strain 1, with 16S RNA Sequence SEQ ID NO:1) was prepared by growing in Hi Veg reinforced clostridial media until early stage stationary phase, and then exchanging with 7.5% Trehalose, 5% Poloxamer, 1% yeast extract, 0.05% cysteine, 20 mM histidine, pH7 via diafiltration. This was frozen at 1° C. per minute followed by lyophilization. The overall titer after lyophilization was reduced by greater than 2 logs from the starting culture. The lyophilized culture was reconstituted in PBS before use.

A healthy fresh culture of Clostridium bolteae was created by inoculating a fresh colony into reduced PYG media and incubating at 37° C. in anaerobic conditions overnight.

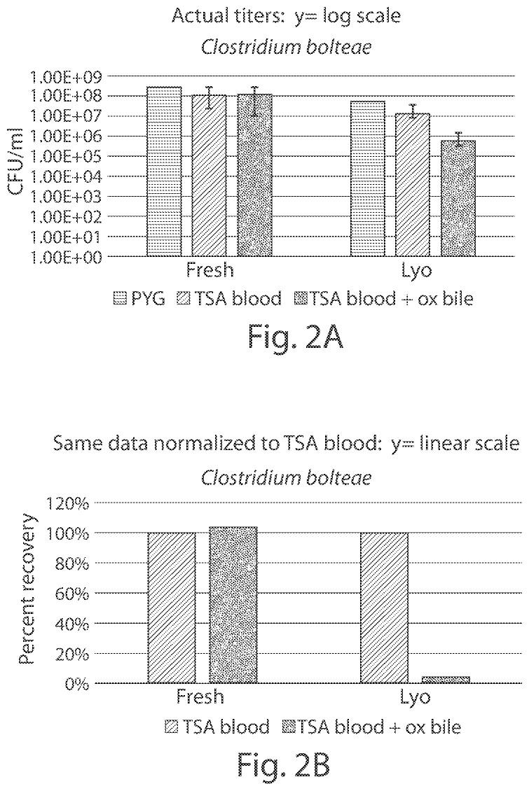

Each of the above Clostridium bolteae cultures were serially diluted in PBS. 100 μl of each of the dilutions were plated in duplicate on pre-reduced Trypticase Soy Agar supplemented with 5% horse blood (TSBA) and pre-reduced TSAB with the addition of 0.15% Ox Bile (the bile acid media). Petri plates were incubated for 3 days at 37° C., and then the colonies were counted and the titers calculated based on the dilutions and plated volume. The results are shown in FIGS. 2A and 2B.

### Example 2: Evaluating Bacterial Integrity Using Selective Media Following Lyophilization

Viability of bacterial compositions was assessed prior to and after lyophilization and storage. In preparing bacterial compositions, for example for use in pharmaceutical compositions for therapeutic applications, the bacterial compositions are cultured and subjected to a lyophilization step, then stored at −80° C., which may impact the viability of the cells. The viability of bacterial compositions prior to and after lyophilization was assessed to determine whether preparation for the lyophilized composition results in a difference in the viability compared to the bacterial compositions prior to lyophilization. Plating the bacterial strains on Ox Bile measures the viability under a stress condition, exposure to bile salts.

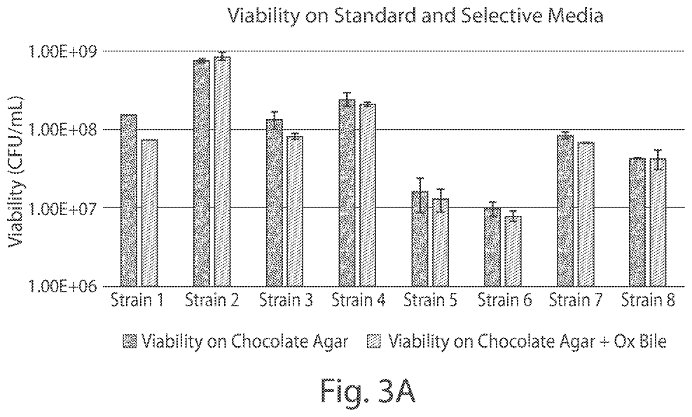

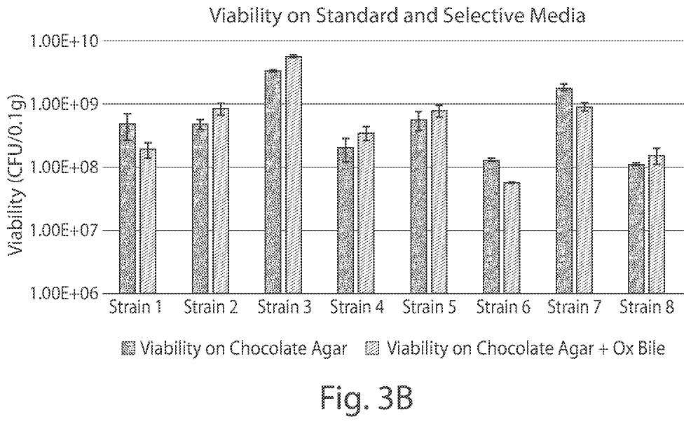

The viability of the bacterial strains (Strains 1-8) on non-selective media (Chocolate Agar) and selective media (Chocolate Agar plus Ox Bile) prior to lyophilization is presented in FIG. 3A, and the viability of the bacterial strains (Strains 1-8) on non-selective media (Chocolate Agar) and selective media (Chocolate Agar plus Ox Bile) following lyophilization is presented in FIG. 3B.

### Example 3: Evaluating Bacterial Integrity Using Selective Media Following Storage

Viability of bacterial compositions was assessed prior to and after storage at 25° C. Bacterial compositions, for example in pharmaceutical compositions for therapeutic applications, may be stored, for example at 25° C. (room temperature) prior to administration to a subject, which may impact the viability of the cells. The viability of bacterial compositions that had been lyophilized was assessed after various lengths of time at 25° C.

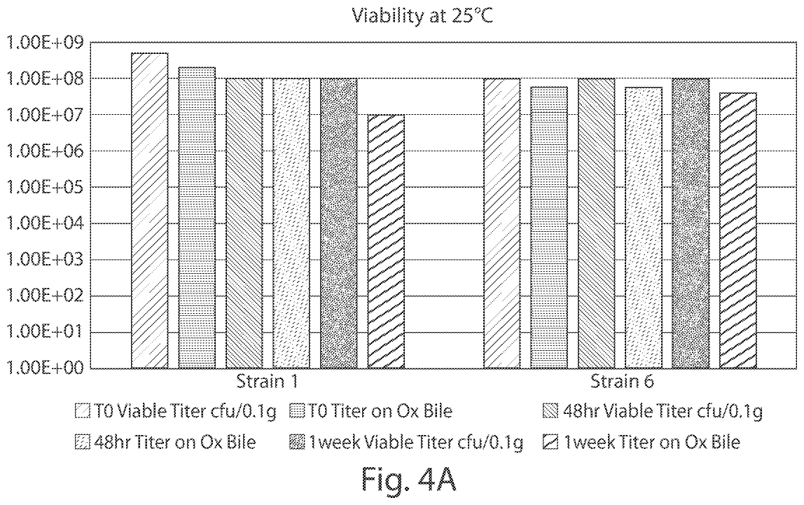

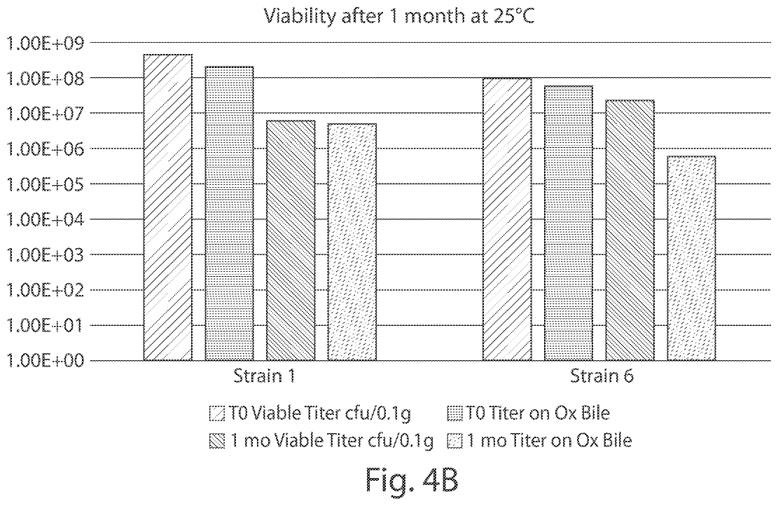

FIG. 4A shows viability of bacterial Strain 1 (Clostridium bolteae) and Strain 6 (Dorea longicatena) on non-selective media and selective media (containing Ox Bile) following storage for 48 hours or one week at 25° C. FIG. 4B shows viability of bacterial Strain 1 (Clostridium bolteae) and Strain 6 (Dorea longicatena) on non-selective media and selective media (containing Ox Bile) following storage for one month at 25° C.

1. A method for determining the integrity of a bacterial composition, the method comprising: growing the bacterial composition on a selective medium, wherein the bacterial composition comprises a bacterial strain, and
wherein if the bacterial composition grows slower on the selective medium than on a non-selective medium, the integrity of the bacterial composition is compromised.

2. The method of claim 1, further comprising growing the bacterial composition on the non-selective medium.

3. The method of claim 1, wherein the bacterial composition was lyophilized prior to growing the bacterial composition.

4. The method of claim 1, wherein if the integrity of the bacterial composition is compromised, the bacterial composition is not used to prepare a live bacterial product.

5. A method for determining the integrity of a first bacterial composition, the method comprising: growing the first bacterial composition on a selective medium, wherein the first bacterial composition comprises a bacterial strain;
growing a second bacterial composition on the selective medium, wherein the second bacterial composition comprises a bacterial strain, and
wherein if the first bacterial composition grows slower than the second bacterial composition, the integrity of the first bacterial composition is compromised.

6. A method for determining the integrity of a first bacterial composition, the method comprising: growing the first bacterial composition on a selective medium,
growing the first bacterial composition on a non-selective medium, wherein the first bacterial composition comprises a bacterial strain;
growing a second bacterial composition on the selective medium,
growing the second bacterial composition on the non-selective medium, wherein the second bacterial composition comprises a bacterial strain;
wherein if the difference in growth between the selective medium and the non-selective medium for the first bacterial composition is greater than the difference in growth between the selective medium and the non-selective medium for the second bacterial composition, and both the first bacterial composition and the second bacterial composition grow slower on the selective medium than the non-selective medium, the integrity of the first bacterial composition is compromised.

7. The method of claim 5, wherein the first bacterial composition was lyophilized prior to growing the first bacterial composition.

8. The method of claim 5, wherein the second bacterial composition was lyophilized prior to growing the second bacterial composition.

9. The method of claim 1, wherein the selective medium is a bile acid media.

10. The method of claim 5, wherein the first bacterial composition and the second bacterial composition comprise the same bacterial strain.

11. The method of claim 5, wherein if the integrity of the first bacterial composition is compromised, the first bacterial composition is not used to prepare a live bacterial product.

12. The method of claim 1, wherein the bacterial strain is an anaerobic bacterial strain.

13. The method of claim 1, wherein the bacterial strain belongs to the class Clostridia.

14. The method of claim 1, wherein the bacterial strain belongs to the family Clostridiaceae.

15. The method of claim 1, wherein the bacterial strain belongs to the genus Clostridium.

16. The method of claim 1, wherein the bacterial strain is selected from the group consisting of Clostridium bolteae, Anaerotruncus colihominis, Ruminococcus torques, Clostridium symbiosum, Blautia producta, Dorea longicatena, Erysipelotrichaceae bacterium, and Subdolinogranulum spp.

17. The method of claim 16, wherein the bacterial strain is Clostridium bolteae.

18. The method of claim 16, wherein the bacterial strain is Dorea longicatena.

19. The method of claim 1, wherein the bacterial strain comprises a 16S rDNA sequence having at least 97% sequence identity with a nucleic acid sequence selected from the group consisting of SEQ ID NOs: 1-8.


SEQUENCE LISTING (8 sequences)

SEQ ID NO: 1 - DNA, 1530 bp, Clostridium bolteae
atgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 2 - DNA, 1529 bp, Dorea longicatena
aacgagagtttgatcctggctcaggatgaacgctggcggcgtgcttaacacatgcaagtcgagcgaagcacttaagtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 3 - DNA, 1529 bp, Ruminococcus torques
tacgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcgtccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctccttt

SEQ ID NO: 4 - DNA, 1527 bp, Clostridium symbiosum
atgagagtttgatcctggctcaggatgaacgctggcggcgtgcctaacacatgcaagtcgaacgaagcgatttaacggaagttttcggatggaagttgaattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctccttt

SEQ ID NO: 5 - DNA, 1531 bp, Blautia producta
atcagagagtttgatcctggctcaggatgaacgctggcggcgtgcttaacacatgcaagtcgagcgaagcacttaagtggatctcttcggattgaagcttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggagggagctgccgaaggcgggaccgataactggggtgaagtcgtaacaaggtagccgtatcggaaggtgcggctggatcacctccttt

SEQ ID NO: 6 - DNA, 1522 bp, Anaerotruncus colihominis
tcaaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 7 - DNA, 1537 bp, Unknown
  Erysipelotrichaceae bacterium
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcggccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccgatgactggggttaagtcgtaacaaggtatccctacgggaacgtggggatggatcacctccttt

SEQ ID NO: 8 - DNA, 1530 bp, Subdoligranulum  spp
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt